(19) United States
(12) Patent Application Publication
(10) Pub. No.: US 2019/0151853 A1
(43) Pub. Date: May 23, 2019

(54) Droplet Thermal Cycling Reaction (DTCR) Device

(71) Applicant: ACUDX Inc., Cupertino, CA (US)

(72) Inventors: Zhangmin Wang, Mountain View, CA (US); Zhuanfen Cheng, San Jose, CA (US); Xitong Li, Cupertino, CA (US)

(21) Appl. No.: 16/192,408

(22) Filed: Nov. 15, 2018

(30) Foreign Application Priority Data
Nov. 16, 2017 (CN) 201711136383.5

(51) Int. Cl. B01L 7/00; C12Q 1/686; B01L 3/00

(52) U.S. Cl. CPC B01L 7/52; C12Q 1/686; B01L 3/502784; B01L 2300/1861; B01L 2300/0627; B01L 2300/0832; B01L 2300/1827; B01L 2300/0848

(57) ABSTRACT

This disclosure provides a droplet thermal cycling reaction (DTCR) device comprising a helical tubing through which a colloid flows; a pump that drives the flow of the colloid; and one or more temperature control sheets (TCS). The pump is configured to drive the colloid to flow through the helical tubing. Optionally, the pump is connected to either the inlet or the outlet of the helical tubing. The TCS sheets, configured to control temperatures for reactions occurring on the device, can be placed either outside or inside helical tubing, and contain at least two temperature zones so that the colloid flows through the different temperature zones along inside the helical tubing. The DTCR of this disclosure has technical benefits of reducing device complexity, enabling device miniaturization, reducing PCR reaction volume, dropletizing PCR reactions, and reducing the cost of digital PCR.

### RELATED APPLICATION

This application claims priority to the Chinese Patent Application No. 201711136383.5, filed on Nov. 16, 2017. The entire content of said application is herein incorporated by reference for all purposes.

### FIELD

This invention relates to a droplet thermal cycling reaction device.

### BACKGROUND

Current technology of droplet thermal cycling reaction (such as droplet digital PCR reaction) is mostly performed in a test tube incubated on a heating block of a PCR device. The thermal cycling is achieved through cycling the temperatures on heating blocks. After the thermal cycling reaction is complete, a separate detection device is needed for quantitative detection of droplets.

Digital PCR is a technology to quantify the absolute number of nucleic acid molecules. Currently there are two PCR-based methods for quantifying nucleic acids. The first is real-time fluorescent quantitative PCR, which is based on a Ct value—the cycle number where the florescent signal first detected above a threshold. The second is digital PCR, which is an absolute quantification method involving micro-fluidic control or micro droplets generation. Micro-fluidic control or microdroplets generation, commonly used in analytical chemistry research, is a method that partitions diluted nucleic acid solution through a chip to micro-reactions or droplets so that each droplet contains less than or equal to 1 nucleic acid template molecule. Thus after PCR cycling, a droplet with one nucleic acid generates florescent signal, and that with no nucleic acid does not. Therefore the amount of target nucleic acids in the original solution can be quantified.

Currently, digital PCR machines are commercially available. However, these products are large (typically over 50 cm height, 20 cm width, and 20 cm length) and costly, which can be a bottleneck for advancing digital PCR technology and its applications. In addition, upon completion of the PCR reaction, a separate detection instrument is needed for quantitative droplets detection. Thus, current devices have the following disadvantages: long digital PCR reaction time, large device footprint, and are complex and difficult to operate.

### BRIEF DESCRIPTION OF THE DRAWINGS

FIG. 1 is a schematic diagram of a droplet temperature cycling reaction device according to an embodiment of the present disclosure. In order to show the internal structure, the temperature control sheets have been disassembled.

### SUMMARY

In some embodiments, this disclosure provides a droplet thermal cycling reaction (DTCR) device comprising a helical tubing connected to an inlet on one end and an outlet on the opposite end, wherein the helical tubing is configured to flow colloidal droplets; a pump that drives the flow of colloid droplets; one or more temperature-control sheets (TCS); and a droplet detection module (DDM) configured to detect the droplets flowing through the outlet; wherein colloid droplets can be introduced to the helical tubing through the inlet; wherein the pump causes the colloid to flow through the helical tubing; wherein the TCS are placed outside or inside the helical tubing to control the temperatures inside the helical tubing; wherein the TCS contain at least two temperature zones, so that colloid droplets can flow through different temperature zones along the helical tubing

In some embodiments, the pump is connected to either the inlet or the outlet of the helical tubing.

In some embodiments, the device includes a droplet detection module (DDM), where the DDM is set at or near the outlet of the helical tubing for colloidal droplets detection. In some embodiments, the droplet detection module is used to detect or quantify the colloidal droplets. In some embodiments, the DDM is positioned such that it only detects the droplets when the thermal cycling reactions inside the droplets are completed. In some embodiments, the DDM is positioned relative to the helical tubing such that it only detects the droplets in the last round of the helical tubing or detects droplets as they pass through the outlet. In some embodiments, the helical tubing is transparent throughout. In some embodiments, only the portion of helical tubing near the outlet is transparent so that the signal from the droplets flowing through the outlet can be detected by the DDM and the rest of the helical tubing is not transparent. In some embodiments, the last round of the helical tubing is transparent while other rounds are not transparent.

In some embodiments, the TCS form a first hollow columnar body and wherein the helical tubing forms a second hollow columnar body. In some embodiments, the first hollow columnar body surrounds the second hollow columnar body. In some embodiments, the first hollow columnar body is enclosed by the second hollow columnar body. In some embodiments, the TCS are in contact with at least a portion of the outer peripheral surface of the helical tubing. In some embodiments, the TCS are in contact with at least a portion of the inside of the helical tubing.

In some embodiments, after colloidal droplets are introduced into the helical tubing through the inlet, the colloidal droplets can move relative to the TCS. In some embodiments, the TCS remain stationary and after the colloid droplets are introduced into the helical tubing through the inlet, the colloid can move relative to the TCS. In some embodiments, after colloid droplets are introduced to the helical tubing through the inlet, the colloidal droplets remains stationary relative to the helical tubing and the TCS rotate so that the colloidal droplets move relative to the TCS. In some embodiments, the pumping rate of the pump is adjustable so that the flow rate of colloidal droplets, the duration of droplets flow through different temperature zones, and the reaction time within droplets in each temperature zone can be adjusted.

In some embodiments, the shape of the cross section of one or more rounds of the helical tubing is round, oval, or polygonal. In some embodiments, the TCS controls temperature inside the helical tubing through resistive heating or radiative heating. In some embodiments, the TCS comprise one sheet and the sheet includes at least two temperature zones.

In some embodiments, the TCS comprise at least two sheets, and the sheets curve and form a hollow columnar body, where each sheet has its own temperature zone. In some embodiments, the TCS comprise three sheets, and the sheets curve and form a shape of a hollow columnar body, and wherein the curve length of the cross section of each of the first and second temperature control sheets is half of the curve length of the cross section of the third temperature-control sheet. In some embodiments, the TCS comprise three sheets, and the sheets curve and form a shape of a hollow columnar body, wherein the curve length of the cross section of the first temperature control sheet is ¼ of the perimeter of the cross section of the hollow columnar body formed by the TCS, wherein the curve length of the cross section of the second temperature control sheet is ¼ of the perimeter of the cross section of the hollow columnar body formed by the TCS, and wherein the curve length of the third temperature control sheet is ½ of the perimeter of the cross section of the hollow columnar body formed by the TCS. In some embodiments, the cross section of the hollow columnar body formed by the one or more temperature-controlling sheets has a diameter of 5-100 mm and a height of 5-100 mm.

In some embodiments, the disclosure provides a method for performing a thermal cycling reaction comprising the steps of: introducing colloidal droplets containing reagents for the thermal cycling reaction to helical tubing of any of the DTCR devices described herein, performing the thermal cycling reaction, and detecting colloidal droplets in which thermal cycling reactions produce detectable signal. In some embodiments, the thermal cycling reaction is a digital PCR. In some embodiments, the method further comprises preparing the colloidal droplets by mixing a first liquid and a second liquid, wherein the second liquid is immiscible with the first liquid, wherein the first liquid contains a plurality of target nucleic acid molecules, and one or more reagents for PCR, whereby forming colloidal droplets. The method further comprises introducing the colloidal fluid into the helical tubing via the inlet. In some embodiments the average number of target nucleic acid molecules per colloidal droplet is less than 10.

### DETAILED DESCRIPTION

Specific embodiments of the present invention will be described below, it should be pointed out that in order to make a concise description it is not possible to describe all features that are present in the actual implementations of the invention in detail.

It should be also understood that during the actual implementation of invention, as in any project or design project, in order to achieve the specific goals of the developer or meet system-related or business-related restrictions, the developer often makes a variety of specific decisions that may vary from one implementation to another. In addition, it should also be understood that although the efforts made in practicing the invention may be complex and lengthy, but for those of ordinary skill in the art, some changes in design, manufacturing, or production are just conventional technical means, and this is not a basis for considering the contents of this disclosure as not sufficient.

Unless otherwise defined, technical or scientific terms used in the claims and description should be the general meaning understood by those with common skills in the technical field to which the present invention belongs.

The terminology used herein is for the purpose of describing particular embodiments only and is not intended to be limiting. As used herein, the singular forms “a,” “an” and “the” are intended to include the plural forms as well, unless the context clearly indicates otherwise. Thus for example, “a temperature control sheet” includes “temperature control sheets” as well.

The “first” and “second” used in the specification and the claims of the disclosure do not indicate any order, quantity, or importance, but only indicate that they are different parts.

“One” does not mean that the number is limited; it means there is at least one.

“Includes” or “contains” refers to that the elements or objects preceding the term “includes” or “contains” covers the elements or objects after the term “includes” or “contains”; the term does not exclude other components or objects.

The words “connected” and the like are not limited to physical or mechanical connections, nor are they limited to direct or indirect connections.

The term “TCS” refers to one or more temperature-control sheets.

The term “a temperature zone” refers to the region on the TCS or the helical tubing that is maintained at a predetermined temperature during a thermal cycling reaction. Different temperature zones are typically maintained under different temperatures during the thermal cycling reactions.

The term “droplet” or “colloidal droplet” refers to a volume of liquid (“first liquid”) formed by distributing the first liquid in small globules in the body of a second liquid, the second liquid being immiscible with the first liquid. Droplets disclosed herein may, for example, be aqueous or non-aqueous or may be mixtures or emulsions including aqueous and non-aqueous components. Droplets may take a wide variety of shapes; non-limiting examples include generally disc shaped, slug shaped, truncated sphere, ellipsoid, spherical, partially compressed sphere, hemispherical, ovoid, and cylindrical shaped.

A goal of this invention is to overcome the disadvantages of existing droplet thermal cycling reaction devices and provide a new droplet thermal cycling reaction device that reduces the complexity of the device, enables the miniaturization of the device, and allows a PCR reaction to be carried out in a smaller volume, dropletizes PCR reaction, thus reducing the cost of digital PCR tests.

The goals of this invention can be achieved through a droplet thermal cycling reaction (DTCR) device. This DTCR device includes a helical tubing for colloidal flow, a pump driving the microfluidic flow, and one or more temperature-control sheets (also referred to as thermostatic sheets).

Materials that are suitable for use as the one or more temperature-control sheets can be any materials that the temperature of which can be adjusted, e.g., by heating. These materials are well known to one of skilled in the art. Examples of such materials include but are not limited to, metal, carbon, silicon, and porcelain. Exemplary methods of controlling the temperature of the TCS include, but is not limited to, using resistive heating or radiative heating to heat the TCS until the temperature reach a predetermined temperature that is suitable for the thermal cycling reaction. Since the TCS are in direct contact with the helical tubing or are in close proximity to the helical tubing, the temperature inside the helical tubing can also be controlled via heat transfer between the one or more temperature-control sheets and the contents (e.g., reaction mixtures) inside the helical tubing.

Said TCS is placed outside or inside the said helical tubing, for colloidal flow and comprises at least two different temperature zones, which allows the colloid to flow through different temperature zones while flowing inside the helical tubing.

Accordingly, this invention disclosed herein advantageously reduces device's complexity and size, reduces the volume of PCR reaction that allows PCR reactions to be performed in droplets, and reduces the cost of digital PCR tests. Specifically, using the existing technology, a droplet thermal cycling reaction is typically carried out in test tubes that are in contact with heating blocks inside a PCR device. The process of temperature cycling for droplets is complex and it is difficult to cyclically adjust the temperature of the heating block. Therefore the existing device used for performing droplets thermal cycling reactions is large, complex, and hard to reduce the size. By comparison, the DTCR device of this invention utilizes the movement of colloid inside a tubing relative to the one or more temperature-control sheets by flowing along a helical path through different temperature zones to achieve thermal cycling. This method allows smaller volume of PCR reaction, e.g., performing the PCR reactions in droplets (dropletizes PCR reaction), and reduces the cost of digital PCR tests. The complexity and the size of the device can be reduced so that it is possible to manufacture the device in the form of a hand-held device having a size close to that of a mobile phone.

In some embodiments, the DTCR device disclosed herein includes a droplet detection module. Such droplet detection module is placed at or near the outlet of the helical tubing for detection of colloidal droplets that produce detectable signals.

Accordingly, this invention's DTCR device advantageously integrates the droplet detection module into the device and thus a separate droplet detection module is not needed for the assay. As such the method further reduces the footprint of the device and shortens the detection time.

In some embodiments, the DTCR device is a quantitative droplet detection module for quantitative detection of colloidal droplets.

Using the above technical method, the DTCR device disclosed herein technically has the benefit of using the quantitative droplet detection module for quantitative detection of colloidal droplets.

In some embodiments, the TCS form a first hollow columnar body (e.g., a hollow cylinder) and the helical tubing forms a second hollow columnar body, and the second hollow columnar body is inside the first columnar body, such that the TCS surrounds the helical tubing.

Using the above technical method, this invention's DTCR device technically has the following advantages: TCS are optimally placed to reduce the device complexity and enable easy and rapid adjustment of the temperatures of the temperature zones thus enable easy and rapid adjustment of thermal cycling of droplets.

In some embodiments, the TCS form a first hollow columnar body and the helical tubing forms a second hollow columnar body and a first hollow columnar body is deposited inside the second hollow columnar body, such that the helical tubing surrounds the TCS.

Using the above technical method, this invention's DTCR device technically has the following beneficial effects: TCS are optimally configured to reduce the device complexity and enable easy and rapid adjustment of the temperatures of the temperature zones and thus enable easy and rapid adjustment of thermal cycling of droplets. Comparing to the method of placing TCS outside the helical tubing, the method reduces the device's footprint further.

In some embodiments, the helical tubing is transparent throughout. In some embodiments, only the portion of helical tubing near the outlet is transparent so that the signal from the droplets flowing through the outlet can be detected by the DDM and the rest of the helical tubing is not transparent. In some embodiments, the last round of the helical tubing is transparent while other rounds are not transparent.

In some embodiments, the DTCR is configured that when colloidal droplets are introduced into the device, the colloidal droplets move relative to the TCS.

Using the above technical method, this invention's DTCR device technically has the following beneficial effects: reducing the device complexity, enabling easy and rapid adjustment of the temperatures of the temperature zones, and thus enabling easy and rapid adjustment of thermal cycling of droplets.

In some embodiments, said TCS is stationary relative to the helical tubing, whereas after colloidal droplets are introduced into the device, said colloidal droplets flow in the helical tubing and move relative to said TCS.

Using the above technical method, this invention's DTCR device uses a more rational droplets movement mechanism to achieve easy and rapid adjustment of the temperatures of the temperature zones and thus reduces device complexity enable easy and rapid adjustment of thermal cycling of droplets.

In some embodiments, after the colloidal droplets are introduced into the device, said colloidal droplets are stationary while the said TCS rotates, resulting that the colloidal droplets move relatively to the said TCS.

Using the above technical method, this invention's DTCR device uses a more optimal droplets movement mechanism to achieve easy and rapid adjustment of the temperatures in the various temperature zones, and thus reduces device complexity and enables easy and rapid adjustment of thermal cycling of droplets.

In some embodiments, the shape of the cross section of each round of the said helical tubing is round, oval, or polygonal.

Using the above technical method, this invention's DTCR device has the following beneficial technical effects: using a more rational shape of each round of the said helical tubing to realize rapid droplets cycling through different temperature zones, reduce the device complexity, thus reduce the device size further.

In some embodiments, the heating method of the said TCS is resistive heating or radiative heating for temperature control. Using the technical method, this invention's DTCR device has the following technical benefits: using a more efficient heating method to enable easy and rapid adjustment of the temperatures of the temperature zones, thus enabling easy and rapid adjustment of thermal cycling of droplets.

In some embodiments, the said pump rate of the pump that drives the colloid flow is adjustable, thus the said flow rate of colloid is adjustable, thus the reaction time of colloidal droplets flowing through each temperature zone is adjustable.

Using the above technical method, this invention's DTCR device technically has the following beneficial effects: the rate of colloid flowing through the different temperature zones is adjustable, thus the reaction time of colloidal droplets within each temperature zone is also adjustable.

In some embodiments, the TCS comprise one temperature-controlled sheet, and the said temperature controlled sheet contains at least two temperature zones.

Using the above technical method, this invention's DTCR device technically has the following beneficial effects: using one temperature controlled sheet to form at least two temperature zones, which simplifies the structure of TCS.

In some embodiments, the TCS comprise at least two temperature-control sheets, and the said temperature controlled sheets form a shape of a hollow cylinder with each temperature-control sheet has its own temperature zone.

Using the above technical method, this invention's DTCR device has the following beneficial technical effects: using one temperature controlled sheet to form at least two temperature zones, thus simplifying the structure of TCS.

In some embodiments, the device comprises three temperature-control sheets, which together form a hollow cylinder, with the first and second sheets each being ¼ cylindrical shape in circumferential direction, and the third sheet being ½ cylindrical shape in circumferential direction, whereby the three temperature-control sheets being assembled in circumferential direction into a hollow cylinder.

Accordingly, the DTCR disclosed herein has the following beneficial technical advantage of optimally arranging the one or more temperature-control sheets, which can adjust temperatures of the reactions fast and conveniently, therefore efficiently regulate the movement and reactions in the colloidal droplets.

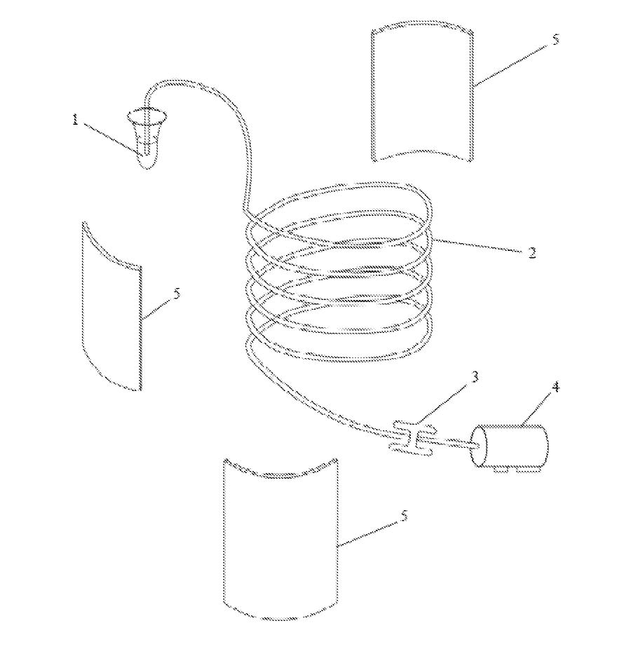

FIG. 1 is an illustration of a non-limiting embodiment of the DTCR of the invention. In order to show the inner structure, the one or more temperature-control sheets are dissembled from the device. The DTCR, as shown in FIG. 1, comprises a helical tubing, a pump, and one or more temperature-control sheets. The reference numerals represent the following:
1: colloidal droplets
2: helical tubing
3: Droplet Detection module (DDM)
4. Pump
5. temperature-control sheets

The colloid comprising the colloidal droplets is connected to the inlet of the helical tubing 2. A pump 4 is connected to the outlet of the helical tubing 2 to drive the colloid through the helical tubing. Although not shown in the FIGURE, one of skilled in the art would readily appreciate that the pump 4 can also be connected to the inlet of the helical tubing, so long as the pump can cause the colloid to flow through the helical tubing 2.

In some embodiments, the one or more temperature-control sheets are in contact with the inside of the helical tubing. In some embodiments, the one or more temperature-control sheets are in contact with the outer peripheral surface of the helical tubing. In some embodiments, the one or more temperature-control sheets are not in contact with the helical tubing, but is in close proximity to the helical tubing, i.e., the closet distance between the one or more temperature-control sheets and the helical tubing is less than 200 μm, less than 100 μm, less than 50 μm, less than 20 μm, or less than 10 μm. The one or more temperature-control sheets comprise at least two different temperature control zones, therefore, when the colloid comprising the colloid droplets flow through the helical tubing, the droplets flow through different temperature-control zones.

Accordingly, the DTCR disclosed herein have the following technical advantages of reducing complexity and size of the equipment, minimize required PCR reaction volumes, droplteize PCR and reduce cost for performing digital PCR.

In some embodiments, the DTCR device includes a colloid droplet detection device 3. The droplet detection device 3 is placed at or near the outlet end of the helical tubing 2 for detecting colloidal droplets.

According to the above technical solution, the DTCR device of the present invention can provide the following benefits. The droplet detection device is also integrated in the device, eliminating the need for additional detection equipment for droplet detection, thus further reducing the equipment size and shortening the detection time.

In some embodiments, the droplet detection device is a droplet quantification device for colloidal droplets. In some embodiments, the droplet quantification device detects colloidal droplets in which thermal cycling reactions produce detectable signal.

According to the above technical solution, the DTCR device of the present disclosure can provide the benefits of quantitatively detecting the colloidal droplets through the liquid droplet quantitative detection device, e. g., a high speed charge-coupled device (CCD) camera or complementary-metal-oxide semiconductor (CMOS) droplet detection device.

In some embodiments, colloidal droplets can be formed in the following manner: mixing two liquids of different properties, one of which forms colloidal droplets in another by surface tension. Typically, an aqueous phase liquid can be used to form colloidal droplets in an oil phase liquid. In some embodiments, colloidal droplets can be formed using a colloidal droplet formation device. In some embodiments, the colloidal droplets contain mixtures, e.g., mixtures of nucleic acids, polymerases, buffer, etc., for thermal cycling reactions.

In some embodiments, the helical tubing is spirally wound for several rounds to form a columnar body. For example, the diameter of the helical tubing may be in the range of 20-500 μm, e.g., about 20-250 μm, 50-150 μm, 60-120 μm, or about 100 μm. In some embodiments, the columnar body consists of 5-500 rounds of tubings, e.g., 10-300, 20-400, 15-100, 20-60, or about 40 rounds., In some embodiments, the formed columnar body has a diameter of 5-100 mm, e.g., 10-60 mm, 25-60 mm, 25-50 mm, or about 30 mm. In some embodiments, the columnar body formed by the helical tubing has a height of 5-300 mm, e.g, 10-200 mm, 20-100 mm, 30-50 mm, or about 40 mm.

In some embodiments, the temperature control range of each temperature control sheet may be 25° C.-99° C., e.g., 40° C.-99° C. or 55° C.-96° C.

In some embodiments, the droplet detection device can use high speed charge-coupled device (CCD) camera or complementary-metal-oxide semiconductor (CMOS) droplet detection device. Detection devices that can be used herein are commercially available, for example, from FLIP Integreated Imaging solutions, Inc. (Richmond, BC, Canada).

In some embodiments, the temperature-control sheets form a hollow columnar body (e.g., a hollow cylinder) and is placed outside and surrounds the hollow columnar body formed by the helical tubing 2. In some embodiments, the TCS are in contact with at least a portion of the helical tubing. According to this technical solution, the DTCR device of the present invention can provide the following benefits: the temperature control piece can be arranged more properly, thereby reducing the complexity of the entire device, and the temperature of the temperature control zone can be conveniently and quickly adjusted, thereby facilitating the rapid adjustment of the droplet temperature cycle.

In some embodiments, the temperature-control sheets form a hollow columnar body, placed inside the hollow columnar body formed by helical tubing 2. In some embodiments, the TCS are also in contact with at least a portion of the helical tubing.

In some embodiments, the diameter of the hollow columnar body formed by the TCS has a diameter of 5-100 mm, e.g., 10-60 mm, 25-60 mm, 25-50 mm, or about 30 mm. In some embodiments, the columnar body formed by the TCS has a height of 5-300 mm, e.g., 10-200 mm, 20-100 mm, 30-50 mm, or about 10-80 mm, 20-60 mm, 30-50 mm, or about 40 mm.

In some embodiments, the height of the hollow columnar body formed by the TCS is 10%-120%, e.g., 10%-40%, 40%-100%, 50%-80%, or 80%-100% of the height of the columnar body formed by the helical tubing. and therefore the temperature control sheets align with the hollow column body formed by the helical tubing.

In some embodiments, the diameter of the hollow columnar body formed by the TCS is substantially similar to that of the diameter of the columnar body formed by the helical tubing, i.e., the diameter of the hollow columnar body formed by the TCS is 80%-120%, e.g., 90%-110%, or 95%-105% of the diameter of the columnar body formed by the helical tubing.

In some embodiments, the hollow columnar body formed by the TCS surrounds the helical tubings. In these embodiments, the diameter of the hollow columnar body formed by the TCS may be larger (e.g., 0-20%, or 0-10% larger) than the hollow columnar body formed by the helical tubing. In some embodiments, the hollow columnar body formed by the TCS is placed inside the helical tubings and is enclosed by the columnar body formed by the helical tubing, and the diameter of the hollow columnar body formed by the TCS is smaller (e.g., 0-20%, or 0-10% smaller) than the diameter of the hollow columnar body formed by the TCS.

According to this technical solution, the DTCR device of the present invention can provide the following benefits. The temperature-control sheets can be arranged properly, thereby reducing the complexity of the device, and the temperature can be adjusted quickly and conveniently. The zone temperature is controlled to facilitate quick and rapid adjustment of the droplet temperature cycle. Moreover, as compared with the technical solution in which the temperature control sheet surrounds the helical tubing and is in contact with at least a portion of the helical tubing, the space occupied by the device can be further reduced.

In some embodiments, the colloidal droplets move relative to the TCS. According to this technical solution, the DTCR device of the present invention can provide the following benefit. The effect of the operation is to reduce the complexity of the device, and the temperature of the temperature control zone can be conveniently and quickly adjusted, thereby facilitating the rapid adjustment of the droplet temperature cycle.

In some embodiments, the temperature control sheet is stationary while the colloidal droplets moves, so that the colloidal droplets moves relative to the temperature-control sheet 5.

According to the above technical solution, the DTCR device of the present invention can provide the following benefits. Through a reasonable method of movement of droplets, the complexity of the equipment is reduced, and the temperature of the temperature control zone can be conveniently and quickly adjusted, thereby facilitating the rapid adjustment of the droplet temperature cycle.

In some embodiments, the colloidal droplets are stationary while the temperature control sheet rotates so that the colloidal droplets moves relative to the temperature control sheet.

According to the above technical solution, the DTCR device of the present invention can provide the following benefits. Through another relatively reasonable method of relative movement of the droplets, the complexity of the equipment is reduced, and the temperature of the temperature control zone can be conveniently and quickly adjusted, thereby facilitating the rapid adjustment of the droplet temperature cycle.

In some embodiments, the shape of cross section of helical tubing 2 is circular, elliptical or polygonal shape.

According to the above technical solution, the DTCR device of the present invention can provide the following benefits. Through the more reasonable shape of each circle of the helical tubing, the rapid circulation of droplets in different temperature control zones can be achieved, the complexity and the size of the device can be further reduced.

In some embodiments, the temperature control sheet 5 uses resistive heating or radiative heating to achieve temperature control.

According to the above technical solution, the droplet temperature cycle reaction device of the present invention can provide the following benefits. Through a more reasonable heating method, the temperature of the temperature control zone can be conveniently and quickly adjusted, so that the droplet temperature cycle can be conveniently and quickly adjusted.

In some embodiments, the pumping speed of the droplet flow driving pump 4 is controllable to control the flow rate of the colloidal droplets, which in turn, controls the reaction time of the colloidal droplets flowing through each temperature control zone.

According to the above technical solution, the DTCR device of the present invention can provide the following benefits. It can control the flow rate of colloidal droplets and control the reaction time of colloidal droplets flowing through each temperature control zone.

In some embodiments, the TCS 5 is a single temperature control sheet, and the single temperature control sheet includes at least two temperature control zones, which can maintain different temperatures.

According to the above technical solution, the DTCR device of the present invention can provide the following benefits. A single temperature control sheet is used to form at least two different temperature control zones, and the temperature control sheet configuration is thus simplified.

In some embodiments, the temperature control sheet has at least two temperature control sheets, which form a hollow columnar body, and each temperature control sheet has its own temperature control zone.

According to the above technical solution, the DTCR device of the present invention can provide the following benefits. At least two temperature control slices are used to form at least two different temperature control zones, which makes it easy to independently adjust the temperatures of the two different temperature control zones.

In some embodiments, the DTCR device comprises three temperature-control sheets, where the three temperature-control sheets curve to form a hollow columnar body. In some embodiments, the curve length of the cross section of each of the first and second temperature control sheets is half of the curve length of the cross section of the third temperature-control sheet. In some embodiments, the cross section of the hollow columnar body is substantially circular and the curve length of the cross section of each of the first temperature control sheet and the second temperature control sheet is ¼ of the perimeter of the cross section of the hollow columnar body formed by the TCS, and the curve length of the third temperature control sheet is ½ of the perimeter of the cross section of the hollow columnar body formed by the TCS.

According to the above technical solution, the DTCR device of the present invention can provide the following benefits. The temperature of the temperature control zone can be conveniently and quickly adjusted through a more reasonable temperature control sheet arrangement, thereby facilitating the rapid adjustment of the droplet temperature cycle.

Of course, the number and shape of the above-mentioned temperature control sheets are only those of the DTCR device of the present application as preferred form, those skilled in the art can understand that based on the disclosure content of the present application, other suitable number and shape of temperature control sheets, for example, two ½ cylinder temperature control sheets, or four cylindrical temperature control sheets, etc., without departing from the scope of the claims of the present application.

The DTCR described herein can be used in a variety of thermal cycling assays. In some embodiments, the DTCR can be used to perform digital PCR. Digital PCR is a quantitative PCR method that provides a sensitive and reproducible way of measuring the amount of DNA or RNA present in a sample. The initial sample mix is partitioned into a large number of droplets prior to amplification step, resulting in either 1 or 0 targets being present in each droplet. Following PCR amplification, the number of positive versus negative reactions is determined and absolute quantification of target can be calculated using Poisson statistics. Unlike real time PCR, in which amplification products are monitored at each cycle of the thermal cycling reaction, the digital PCR reaction are run to endpoint and the presence or absence of the detectable signal, for example, fluorescence, is then used to calculate the absolute number of targets present in the original sample. Droplets with signal are positive and scored as “1”, and droplets with background signal are negatives and scored as “0”. Poisson statistical analysis is then used to determine the absolute concentration of target present in the initial sample.

In one embodiment, the number of the colloidal droplets is greater than 10, 100, 1000, 10000, 100000, or 1000000; the droplet size is less than 1000 nanoliter (“nl”), less than 100 nl, less than 10 nl, less than 1 nl, less than 0.1 nl, less than 0.01 nl, or less than 0.001 nl; so that in one reaction the average number of target DNA molecules per droplet (also called lamda, which is defined as the total number of DNA targets dispersed into all droplets divided by the total number of droplets) is less than 10, 5, 3, 2, 1, 0.5, 0.1, or 0.01. Assuming target DNA molecules are dispersed into droplets through a random process, the number of droplets having detectable signal and the number of those not are used to calculate the original total number of targets through Poisson formula:

$${N}_{t} = {D}_{t} ln ( \frac{{D}_{t}}{{D}_{n}} )$$

where Nₜ represents total number of the target molecules across all droplets, Dₜ the number of total droplets, and Dₙ the number of droplets having no detectable signal.

Some exemplary embodiments have been described above. However, it should be understood that one can make various modifications. For example, if the described techniques are performed in a different order and/or if the components in the described system, architecture, device, or circuit are combined in different ways and/or replaced or supplemented by other components or their equivalents, one can still achieve suitable results. Accordingly, other embodiments shall also be within the scope of the claims.

### NON-LIMITING EXEMPLARY EMBODIMENTS

This disclosure includes the following non-limiting exemplary embodiments:

1. A droplet thermal cycling reaction (DTCR) device comprises a helical tubing is connected to an inlet on one end and an outlet on the opposite end, wherein the helical tubing is configured to flow colloidal droplets, a pump that drives the flow of colloid droplets; and one or more temperature-control sheets (TCS);
where colloid droplets can be introduced to the helical tubing through the inlet;
where the pump is connected to either the inlet or the outlet of the helical tubing;
wherein the pump causes the colloid to flow through the helical tubing;
where the TCS are placed outside or inside the helical tubing; and
where the TCS contain at least two temperature zones, so that colloid droplets can flow through different temperature zones along inside the helical tubing.
2. The device of embodiment 1, wherein the device includes a droplet detection module (DDM), where the DDM is located at or near the outlet of the helical tubing for colloidal droplets detection.
3. The device of embodiment 2, wherein the droplet detection module is used to detect or quantify the colloidal droplets.
4. The device of any of embodiments 1-3, wherein the TCS form a first hollow columnar body and wherein the helical tubing forms a second hollow columnar body.
5. The device of embodiment 4, wherein the first hollow columnar body surrounds the second hollow columnar body.
6. The device of embodiment 4, wherein the first hollow columnar body is enclosed by the second hollow columnar body.
7. The device of embodiment 5, wherein the TCS are in contact with at least a portion of the outer peripheral surface of the helical tubing.
8. The device of embodiment 6, wherein the TCS are in contact with at least a portion of the inside of the helical tubing.
9. The device of any of embodiments 1-8, wherein after colloid droplets are introduced into the helical tubing through the inlet, the colloidal droplets can move relative to TCS.
10. The device of any of embodiments 1-8, wherein the TCS remain stationary and after the colloid droplets are introduced into the helical tubing through the inlet, the colloid can move relative to the TCS.
11. The device of any of embodiments 1-8, wherein after colloid droplets are introduced to the helical tubing through the inlet, the colloidal droplets remains stationary relative to the helical tubing and the TCS rotate so that the colloidal droplets move relative to the TCS.
12. The device of any of embodiments 1-11, wherein the shape of the cross section of one or more rounds of the helical tubing is round, oval, or polygonal.
13. The device of any of embodiments 1-12, wherein the TCS controls temperature inside the helical tubing through resistive heating or radiative heating.
14. The device of any of embodiments 1-13, wherein the pumping rate of the pump is adjustable so that the flow rate of colloidal droplets, the time of droplets flow through different temperature zones, and the reaction time within droplets in each temperature zone can be adjusted.
15. The device of any of embodiments 1-14, wherein the TCS comprise one sheet and the sheet includes at least two temperature zones.
16. The device of any of embodiments 1-14, wherein the TCS comprise at least two sheets, and wherein the sheets curve and together they form a shape of a hollow columnar body, where each sheet has its own temperature zone.
17. The device of any of embodiments 1-14, wherein the TCS comprise three sheets, and wherein the sheets curve and together they form a shape of a hollow columnar body, and wherein the length of the cross section of each of the first and second temperature control sheets is half of the length of the cross section of the third temperature-control sheet.
18. The device of any of embodiments 1-14, wherein the TCS comprise three sheets, and wherein the sheets curve and together they form a shape of a hollow columnar body,
wherein the curve length of the cross section of the first temperature control sheet is ¼ of the perimeter of the cross section of the hollow columnar body formed by the TCS,
wherein the curve length of the cross section of the second temperature control sheet is ¼ of the perimeter of the cross section of the hollow columnar body formed by the TCS, and
wherein the curve length of the third temperature control sheet is ½ of the cross section of the hollow columnar body formed by the TCS.
   19. The device of any of embodiments 4-18, wherein the hollow columnar body formed by the one or more temperature-controlling sheets has a diameter of 5-100 mm and a height of 5-100 mm.
   20. A method for performing a thermal cycling reaction comprising the steps of:
adding colloidal droplets comprising reagents for the thermal cycling reaction to the droplet thermal cycling reaction device of any of embodiments 1-19, and
performing the thermal cycling reaction.
   21. The method of embodiment 20, further comprising detecting colloidal droplets in which thermal cycling reactions produce detectable signal using a droplet detection module (DDM), located at or near the outlet of the helical tubing for colloidal droplets detection.
   22. The method of any of embodiments 20-21, wherein the thermal cycling reaction is a digital PCR.
   23. The method of any of embodiments 20-22, wherein the method further comprises preparing the colloidal droplets by mixing a first liquid and a second liquid, wherein the second liquid is immiscible with the first liquid,
   wherein the first liquid contains a plurality of target nucleic acid molecules, and one or more reagents for PCR, whereby forming colloidal droplets.
   24. The method of any of embodiments 20-23, wherein the average number of target nucleic acid molecules per colloidal droplet is less than 10.

1. A droplet thermal cycling reaction (DTCR) device comprises a helical tubing connected to an inlet on one end and an outlet on the opposite end,
wherein the helical tubing is configured to flow one or more colloidal droplets; a pump that drives the flow of colloid droplets; one or more temperature-control sheets (TCS); and a droplet detection module (DDM) configured to detect the one or more droplets at the endpoint of thermal cycling reactions;
wherein colloid droplets can be introduced to the helical tubing through the inlet;
wherein the pump causes the colloidal droplets to flow through the helical tubing;
wherein the TCS are placed outside or inside the helical tubing to control temperatures inside the helical tubing;
wherein the TCS contain at least two temperature zones, so that colloid droplets can flow through different temperature zones along the helical tubing.

2. The device of claim 1, wherein the device includes a droplet detection module (DDM), where the DDM is at or near the outlet of the helical tubing for colloidal droplets detection such that the DDM can detect droplets flow through the outlet.

3. (canceled)

4. The device of claim 1, wherein the TCS form a first hollow columnar body and wherein the helical tubing forms a second hollow columnar body.

5. The device of claim 4, wherein the first hollow columnar body surrounds the second hollow columnar body.

6. The device of claim 4, wherein the first hollow columnar body is enclosed by the second hollow columnar body.

7. The device of claim 5, wherein the TCS are in contact with at least a portion of the outer peripheral surface of the helical tubing.

8. The device of claim 6, wherein the TCS are in contact with at least a portion of the inside of the helical tubing.

9. The device of claim 1, wherein after colloid droplets are introduced into the helical tubing through the inlet, the colloidal droplets can move relative to TCS.

10. The device of claim 1, wherein the TCS remain stationary and after the colloid droplets are introduced into the helical tubing through the inlet, the colloid can move relative to the TCS.

11. The device of claim 1, wherein after colloid droplets are introduced to the helical tubing through the inlet, the colloidal droplets remains stationary relative to the helical tubing and the TCS rotate so that the colloidal droplets move relative to the TCS.

12. The device of claim 1, wherein the shape of the cross section of one or more rounds of the helical tubing is round, oval, or polygonal.

13. The device of claim 1, wherein the TCS controls temperature inside the helical tubing through resistive heating or radiative heating.

14. The device of claim 1, wherein the pumping rate of the pump is adjustable so that the flow rate of colloidal droplets, the time of droplets flow through different temperature zones, and the reaction time within droplets in each temperature zone can be adjusted.

15. The device of claim 1, wherein the TCS comprise one sheet and the sheet includes at least two temperature zones.

16. The device of claim 1, wherein the TCS comprise at least two sheets, and wherein the sheets curve and together they form a shape of a hollow columnar body, where each sheet has its own temperature zone.

17. The device of claim 1, wherein the TCS comprise three sheets, and wherein the sheets curve and together they form a shape of a hollow columnar body, and wherein the length of the cross section of each of the first and second temperature control sheets is half of the length of the cross section of the third temperature-control sheet.

18. The device of claim 1, wherein the TCS comprise three sheets, and wherein the sheets curve and together they form a shape of a hollow columnar body,
wherein the curve length of the cross section of the first temperature control sheet is ¼ of the perimeter of the cross section of the hollow columnar body formed by the TCS,
wherein the curve length of the cross section of the second temperature control sheet is ¼ of the perimeter of the cross section of the hollow columnar body formed by the TCS, and
wherein the curve length of the third temperature control sheet is ½ of the cross section of the hollow columnar body formed by the TCS.

19. The device of claim 4, wherein the hollow columnar body formed by the one or more temperature-controlling sheets has a diameter of 5-100 mm and a height of 5-100 mm.

20. A method for performing a thermal cycling reaction comprising the steps of:
introducing colloidal droplets containing reagents for the thermal cycling reaction to the droplet thermal cycling reaction device of claim 1, and
performing the thermal cycling reaction, and
detecting colloidal droplets in which thermal cycling reactions produce detectable signal.

21. The method of claim 20, wherein the thermal cycling reaction is a digital PCR.
